(19) United States
(12) Patent Application Publication
(10) Pub. No.: US 2006/0200078 A1
(43) Pub. Date: Sep. 7, 2006

(54) Medical assembly, in addition to a guard device, puncture element and a manipulation device for said assembly

(76) Inventor: Franz Konrad, OBERNDORF bei SCHWANENSTADT (AT)

Correspondence Address: WILLIAM COLLARD;COLLARD & ROE, P.C., 1077 NORTHERN BOULEVARD, ROSLYN, NY, 11576, US

(21) Appl. No.: 10/543,532

(22) PCT Filed: Nov. 19, 2003
(86) PCT No.: PCT/AT03/00345

§ 371 Date: Oct. 13, 2005

(30) Foreign Application Priority Data
Jan. 27, 2003 (AT) A 104/2003

(51) Int. Cl. A61M 5/00

(52) U.S. Cl. 604/110; 600/576; 604/164.08; 128/919

(57) ABSTRACT

The invention relates to an assembly (1) comprising a manipulating device (2), a puncturing element (3) designed for coupling it to said device, and a pivot-mounted protective device (4). A positioning device (26) is arranged between the holding elements (11, 24) of the protective device (4) and the manipulating device (2), whereby said positioning device, with the holding elements (11, 24) being engaged, is fixing a plane of swivel (22) of a protective element (23) of the protective device (4) in its position. Coupling elements (12, 16) of a coupling device (18) disposed between the manipulating device (2) and the puncturing element (3) are designed in relation to each other in such a way that with the two coupling elements (12, 16) in their completely coupled positions, a shorter opening axis (21) of an aperture (19) disposed at the proximal end (13) of the puncturing element (3) is aligned extending about parallel to the plane of swivel (22), or in the plane of swivel (22). Furthermore, the invention relates to the protective device (4), the puncturing element (3), as well as to the manipulating device (2).

The invention relates to an construction assembly particularly for the medical technology, comprising a manipulating device with a first holding element arranged thereon, as well as a first coupling element, and a puncturing element with a proximal end and a distal end, whereby a passage connects the two ends with one another, and another coupling element arranged between said ends, whereby the two coupling elements form a coupling device, and the puncturing element is secured, if necessary detachably on the manipulating device; whereby the proximal end projects beyond the manipulating device, and an aperture with a longer and a shorter opening axis is arranged at least on the proximal end; a pivoting protective device at least for a part area of the puncturing element; and whereby the protective device is secured, if need be in a detachable manner with another holding element on the first holding element of the manipulating device in a stationary manner, and a protective element of the protective device is adapted to pivot from a first position (=the release position) relating the part area, into a second position (=the protective position) covering the part area.

A medical assembly, for example for drawing blood, has become known from U.S. Pat. No. 3,658,061 A, which comprises a manipulating device, a puncturing element held thereon or inserted therein, as well as a pivot-mounted protective device for at least a part area of the puncturing element. The protective device can be detached, if need be, however, it is mounted stationary on the manipulating device, whereby a flexible component disposed between the protective element and the holding element of the protective device is forming a joint arrangement. The protective device is adapted for pivoting from a first position in which it releases the puncturing element, into a second position in which it covers the puncturing element. The protective element has a duct for receiving a part area of the puncturing element, whereby said duct is designed in such a way that when it is in its protective position, the protective element is retained on the puncturing element by friction grip or clamping.

Another protective element has become known from U.S. Pat. No. 4,664,259 A, in connection with which a locking element is associated with the protective element within the range of the latter when said protective element is in the folded-in position, i.e. in the protecting position. With said locking element, the protective element can be kept locked on the puncturing element, particularly a hollow needle, when it is in its protecting position, in order to prevent the proximal end of the puncturing element from being unintentionally released again.

Another protective device for a medical assembly has become known from EP 0 626 924 B1, in connection with which the protective element can be detachably mounted on the manipulating device, if need be. In this connection, the holding element of the protective device is supported in a stationary manner on the manipulating device in the direction of the longitudinal axis, thus in the axial direction on the manipulating device, whereby, however, the holding element with the protective element can be rotated around the longitudinal axis.

Other devices for protecting needles have become known from EP 0 702 973 B1 and EP 0 885 621 B1, where the holding element for the protective element of the protective device is mounted in an area of the puncturing element that is facing away from the manipulating device. A construction unit is provided in this manner in connecting with which the puncturing element and the protective element can be jointly inserted in the manipulating device in most cases. Similarly designed devices for protecting needles have become known also from U.S. Pat. No. 6,436,086 B1, U.S. Pat. No. 6,440,104 B1, US 2002/0151852 A1, US 2002/0151853 A1, US 2002/0156425 A1, and US 2002/0161336 A1.

The drawback afflicting all of said needle protection devices is that the position of the aperture at the proximal end has always been uncertain with respect to the predetermined position of the plane of swivel of the protective element, as this has been the case with the components that can be mounted on the manipulating device independently of one another, namely the protective device and the puncturing element.

The present invention is based on the problem of providing a construction assembly particularly for the medical technology, with a protective device, a puncturing element and a manipulating device for such a construction assembly, in connection with which said components can be mounted independently of each other, permitting safe and mainly trouble-free control when such an assembly is employed as intended.

The problem of the invention is resolved by the totality of the features of claim 1, in particular in that a positioning device is arranged between the to holding elements of the holding device, said positioning device fixing the position of the plane of swivel of the protective elements of the protective device with respect to the manipulating device as the holding elements are being engaged; and that the two coupling elements of the coupling device are designed with respect to one another in such a way that when the two coupling elements are in the position in which they are completely engaging each other, the shorter opening axis of the aperture is aligned extending approximately parallel to the plane of swivel, or in the plane of swivel. The advantage surprisingly ensuing there from is that the position of the plane of swivel of the protective element of the protective device is fixed with respect to the manipulating device by the positioning device arranged between the two holding elements, with said holding elements being engaged. A defined position of the protective element relative to the manipulating device is achieved by virtue of the additional arrangement of the positioning device. Furthermore, by forming the coupling elements between the puncturing element and the manipulating device the position and thus the alignment of the aperture in the area of the proximal end is fixed depending on the positioning device in such a way that the shorter opening axis of the aperture is aligned extending approximately parallel to the plane of swivel, or in the plane of swivel. What is achieved in this manner is that when the assembly is employed in accordance with its intended application, for example for collecting or drawing body fluids, or for dispensing fluids into the body of a patient, the protective element is always arranged laterally with respect to the manipulating device. In this way, an application of the assembly particularly in the medical technology with high user safety is achieved, because owing to the determination of the position between the plane of swivel, such position being determined in cooperation with the positioning device, and to the design of the coupling device, which is subsequently coordinated therewith, it is always avoided that the protective element either faces the patient directly, or that it is made more difficult or impossible for the operator to view the aperture on the proximal end. Furthermore, owing to the predetermined lateral arrangement of the protective element, a simple closing movement of the protective element into the protective position can take place because in its release position, the protective element needs to be pressed only, for example against a suitably stable object, so that the proximal end of the puncturing element can be covered in this way without any risk of causing injury. Moreover, a simple movement for inserting the puncturing element into the manipulating device is achieved in this way, because the cannula can be attached by connecting it without any accessory like the pivotmounted protective device. Another embodiment according to claim 2 is advantageous as well in that an easily manageable manipulating device is provided that favors the reception of suitably designed receiving vessels such as, for example tubes for blood specimens.

Furthermore, the embodiment according to claim 3 is beneficial in that different components can be simply and, most important of all, safely attached or coupled to the manipulating device in this way in the smallest possible space.

The embodiment according to claim 4 or 5 is beneficial in that it permits forming on the manipulating device a component with a simple design that can be ejected or removed from the mold in a simple way, and on which both the puncturing element and the pivot-mounted protective device can be secured in a simple manner.

According to another design variation according to claim 6, mounting of the protective device on the manipulating device is achieved in a simple way due to the design of the tubular components on both sides.

However, the further developments of the construction assembly according to claims 7 to 9 is advantageous as well in that an axial fixation or mounting of the holding element of the protective device on the holding element of the manipulating device is ensured in this manner, and the protective device can be held on the manipulating device in a simple and, if need be, detachable way.

In the embodiment according to claim 10, the advantage gained is that a clearly fixed pivot center is provided in this way for the protective element with respect to the puncturing element held on the manipulating device.

What is achieved with the further development according to claim 11 or 12 is that an easily operable coupling device is provided in this way, where due to the double thread, an end position of the aperture arranged on the proximal end that is pivoted by 180° is achievable with respect to the manipulating device, and the release position of the protective element is fixed in this simple manner as the device is being used in accordance with its intended application.

Owing to the embodiment according to claim 13, the position of the puncturing element is additionally fixed on the manipulating device in connection with the puncturing element used.

The embodiment according to claim 14 or 15 is beneficial as well because the alignment, and connected therewith the position of the plane of swivel with respect to the manipulating device can be fixed in this way in a simple manner. In this way, not only a fixed position of the entire protective device on the manipulating device is achieved, but also an exact, predeterminable alignment of the plane of swivel.

According to the embodiment specified in claim 16, another safe coupling between the puncturing element and the manipulating device can be achieved for the intended application.

The embodiment according to claim 17 is advantageous in that in the protective position, any unintended detachment or release again of the proximal end of the puncturing element is safely prevented in this way.

The advantage gained with the embodiment according to claim 18 is that the puncturing element is completely covered all around by the channel formed in the protective element up to the required receiving aperture, so that high safety is achieved in the protective position for the operating personnel.

According to another design variation according to claim 19, the protective element for the puncturing element is kept locked in its protective position against unintended detachment, which prevents it from being used again and thus prevents injury to the operating personnel and thus also other patients from getting infected.

The design according to claim 20 or 21 is beneficial as well in that due to the adjacent arrangement of the retaining element with respect to the holding element, or in relation to the swivel arrangement such as, for example the film hinge arranged thereon the spacing is selected in such a way that in a new opening attempt, any bending of the puncturing element due the shorter lever effect between the retaining element and the clamping site of the latter is prevented for the most part. With a larger spacing or distance of the retaining element from the holding element or axis of swivel of the protective element, the retaining element may slip out due to the inherent elasticity of the puncturing element, which otherwise would permit it to be used again in a simple manner.

The design according to claim 22 or 23 permits achieving a high degree of coverage of the puncturing element within the protective element, so that the proximal end of the puncturing element is safely covered even if the assembly is improperly operated, or any unscheduled attempt is made to use it again, which means that injury due to puncture and infections connected therewith are avoided.

The problem of the invention, however, is independently resolved as well by the features of claim 24. The advantages ensuing from the combination of features specified in the characterizing part of said claim lie in that a protective device is provided in this way for an assembly, where a predeterminable alignment of the plane of swivel for the protective element is achievable with respect to its mounting on the manipulating device. Owing to such predeterminable position, it is consequently possible to provide through additional coordination of the two coupling elements between the puncturing element and the manipulating device for an alignment of the position in relation to the manipulating device, and connected therewith for a predefined alignment of the plane of swivel.

According to another design variation according to claim 25, provision is made for holding the protective device on the manipulating device in a simple manner by forming the tubular components on both sides.

The embodiment according to claim 26 or 27 is advantageous as well in that the alignment and, connected therewith the position of the plane of swivel with respect to the manipulating device can be fixed thereby in a simple manner. Not only a stationary positioning of the entire protective device is achieved in this manner, but also an exact, predeterminable alignment of the plane of swivel.

According to an advantageous further development as specified in claim 28 or 29, the part area of the puncturing element is safely covered, and, in addition, unintentional release is prevented.

The embodiment according to claims 30 and 31 is advantageous as well in that owing to the fact that the retaining element is arranged adjacent to the holding element, or to the swivel arrangement, e.g. a film hinge arranged on the latter, the spacing is selected in such a way that when a new opening attempt is made, bending of the puncturing element is prevented for the most part due to the shorter lever effect between the retaining element and the clamping site. With a larger spacing or distance of the retaining element from the holding element or the axis of swivel of the protective element, the puncturing element may slip from the retaining element due to its inherent elasticity, which otherwise may permit it to be used again in a simple manner.

Another embodiment as specified in claim 32 is beneficial in that a predeterminable support surface of the retaining element is provided in this manner for the hollow needle forming the puncturing element, which prevents the protective device from being unintentionally swiveled away again, and, in connection therewith, the proximal end of the puncturing element is prevented from being released.

The embodiment according to claim 33 or 34 is beneficial because a high degree of coverage is achievable in this way, and, in connection therewith, high safety for the protective element.

Due to the design according to claim 35, it is possible to cover also the other coupling element of the puncturing element against the bridges of the protective element, so that a high degree of coverage is obtained in that area as well, and, connected therewith, high safety against transmission of infections is achieved in this manner as well.

According to another design variation according to claim 36, high strength and, in connection therewith, inherent rigidity of the protective element are achieved combined with a high degree of coverage.

The embodiment according to claim 37 is beneficial in that the depth of the duct is increased in this manner in the direction of the plane of swivel within the area of the proximal end of the needle to be covered, which provides an additional space for collecting any fluids exiting from the hollow needle, leading in turn to even higher safety for the personnel operating the device.

With the embodiment according to claim 38, it is advantageous that the duct formed in the protective element completely covers the puncturing element except for the required receiving aperture, so that high safety is achievable for the operating personnel in the protective position.

The further development according to claim 39 or 40 leads to achieving high operating safety for the protective element, so that the manipulative quality is enhanced in this manner for the operating personnel.

The embodiment according to claim 41 provides an operating surface on the protective element protruding against the duct aperture, with which said protective element can be set to the protective position without touching it by hand. By supporting this section of the gripping bar on a fixed object, the protective element can be adjusted to the protective position without required the user to use his or her hand in that area.

The design according to claim 42 is beneficial because a simple limitation of the duct is achieved in this manner.

Embodiments as specified in claims 43 to 47 are advantageous as well in that in the even of any incorrect operation, the proximal end is prevented from penetrating the wall part here limiting the duct.

Owing to the embodiment according to claims 48 and 49, it is possible to achieve a high degree of coverage of the puncturing element within the protective element, which, even with any improper operation, ensures that the proximal end of the puncturing element is safely covered should a new attempt be unintentionally made to use the device, so that injury due to puncture and infections connected therewith are prevented from occurring.

According to claim 50, a simple possibility is provided for designing the retaining element.

The embodiment according to claim 51 facilitates the accommodation of the puncturing element toward the bottom of the duct, on the one hand, and its release without manipulation is safely prevented on the other.

It is advantageous in connection with the embodiment according to claim 52 that a clearly fixed pivoting center is provided for the protective element with respect to the puncturing element held on the manipulating device.

The embodiment according to claim 53 is beneficial as well in that it provides a design of the axis of swivel at favorable cost, and at the same time a clear fixation of the swivel point for the protective element.

The embodiment according to claim 54 is advantageous in that prior to the first setting, a defined positioning of the protective element relative to the position of the drawing needle is predetermined in this manner, which in turn permits a safe operation.

The problem of the invention, however, is independently resolved also by the features of claim 55. The advantages ensuing from the combination of features specified in the characterizing part of this claim lie in that a manipulation device is provided in this manner for a medical assembly designed for receiving or coupling a protective device as well as a puncturing element, whereby due to the additional arrangement of the positioning device between the protective device and the manipulating device, as well as due to the alignment of the coupling device between the puncturing element and the manipulating device, the position of the aperture arranged at the proximal end of the puncturing element in relation to the plane of swivel of the protective element can be clearly predetermined, and safe handling is assured when such an assembly is employed in accordance with its intended application.

Another embodiment according to claim 56 is beneficial as well because a manipulating device is provided in this way that can be easily managed, and is favoring the reception of suitably designed receiving vessels such as, for example tubes for blood specimens.

Furthermore, an embodiment according to claim 57 is beneficial because it permits different components to be mounted on or coupled to the manipulating device in a simple and, most important of all, safe manner in the smallest of space.

Owing the embodiment according to claim 58 or 59, it is possible to arrange on the manipulating device a component that has a simple design and can be easily removed or ejected from the mold, and on which both the puncturing element and the pivoting protective device can be mounted or held in a simple manner.

By virtue of the further development according to claim 60 or 61, a coupling device is obtained that can be easily operated, and in connection with which, due to the double thread, it is possible to achieve an end position of the aperture at the proximal end that is offset by 180° relative to the manipulating device, so that the release position of the protective element can be simply fixed in this manner as the assembly is being employed in accordance with the application for which it is intended.

The embodiment according to claim 62 or 63 is advantageous as well in that the alignment and, in connection therewith the position of the plane of swivel in relation to the manipulating device can be fixed in a simple manner. No only a fixed position of the entire protective device on the manipulating device is obtained in this way, but also an exact, predeterminable alignment of the plane of swivel is achieved.

The problem of the invention is independently resolved also by the features of claim 64. The benefits resulting from the combination of features specified in the characterizing clause of this claim lie in that a puncturing element such as a hollow needle, is provided for an assembly, in connection with which a predeterminable alignment of the aperture on the hollow needle in relation to the plane of swivel of the protective element on the manipulating device is achievable. In this way, a predeterminable position can be always achieved between the individual components for their employment, and thus their safe use in the application intended for such components.

The objective achieved with the further development according to claim 65 or 66 is that a coupling device that can be easily operated is obtained in this manner, whereby due to the double thread, an end position of the aperture on the proximal end is offset by 180° in relation to the manipulating device, so that the release position of the protective element is simply fixed in this manner as the assembly is being used in accordance with the application for which it is intended.

Finally, according to the embodiment specified in claim 67, another safe coupling between the puncturing element and the manipulating device is realized for its use in accordance with the intended application.

The invention is explained in greater detail in the following with the help of the exemplified embodiment shown in the drawings, in which:

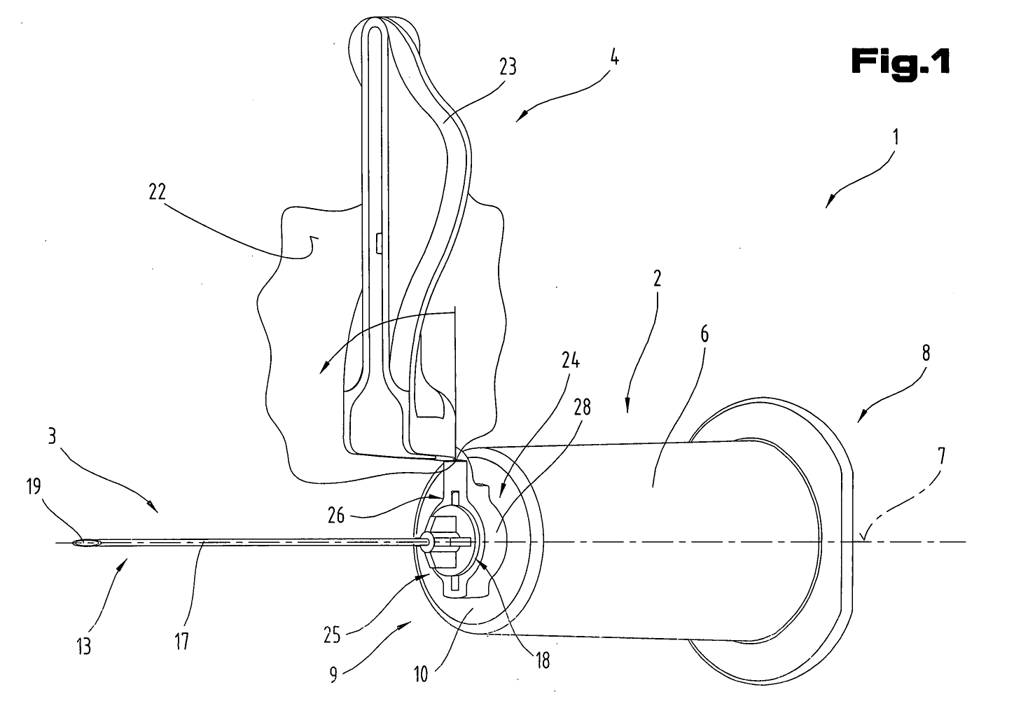

FIG. 1 shows a simplified perspective view of an assembly as defined by the invention.

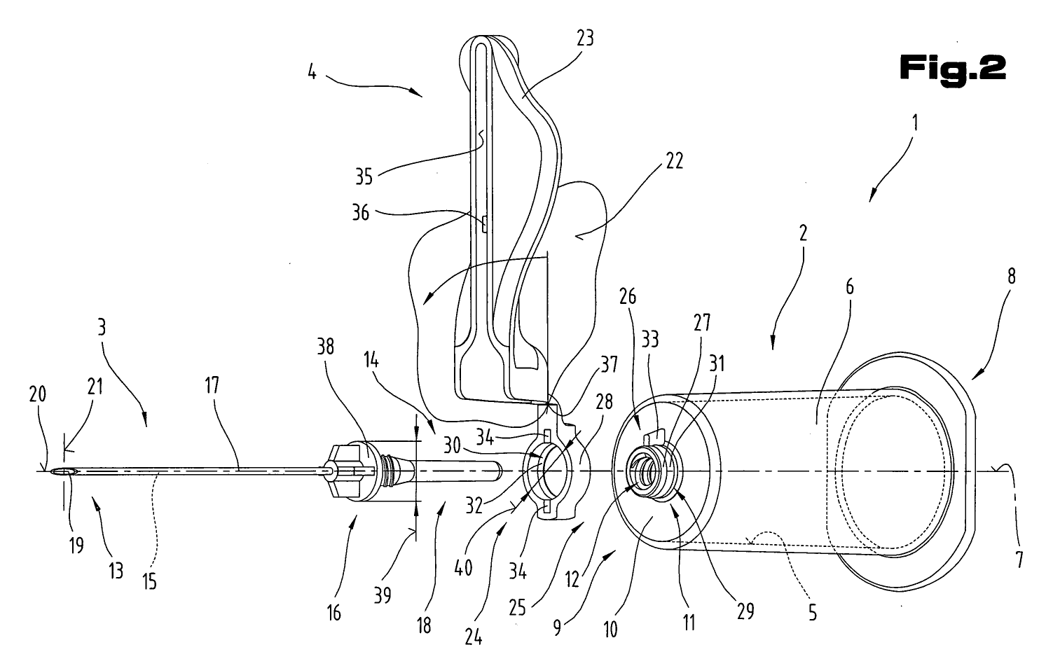

FIG. 2 shows a simplified representation of the assembly according to FIG. 1, where the individual components are spaced from one another.

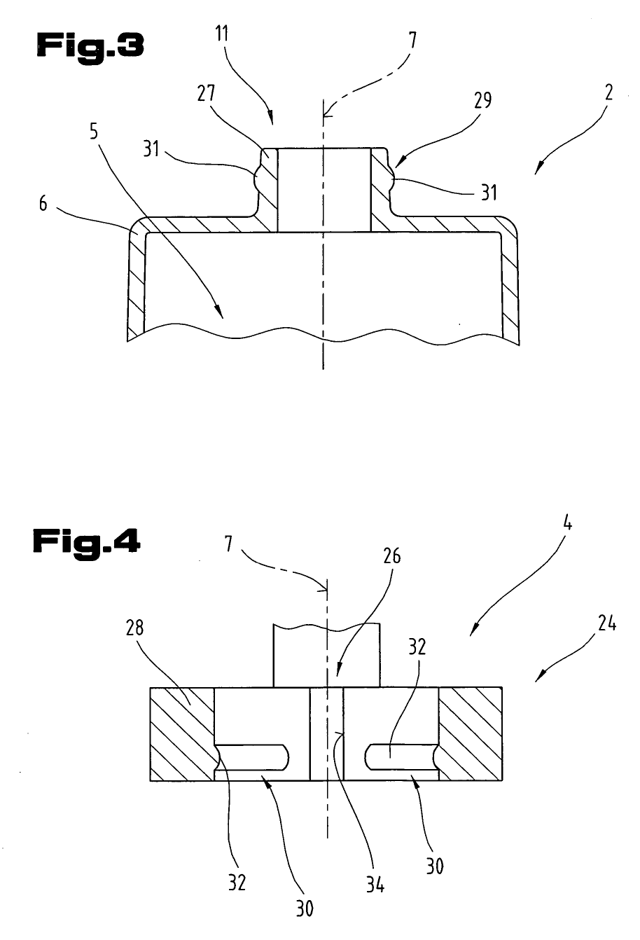

FIG. 3 is a sectional side view and simplified, enlarged representation of a part area of the manipulating device in the area of the first holding element.

FIG. 4 is a sectional view and simplified, enlarged representation of a part area of the protective device in the area of the other holding element.

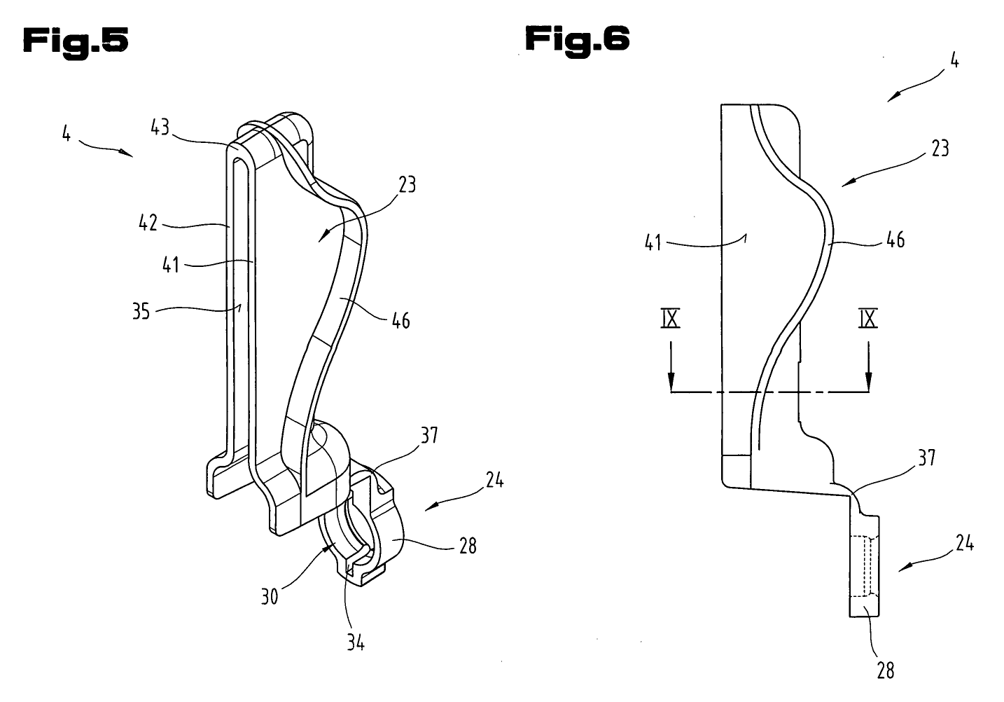

FIG. 5 shows a simplified perspective representation of another protective device as defined by the invention for forming the assembly.

FIG. 6 is a view of the protective device according to FIG. 5.

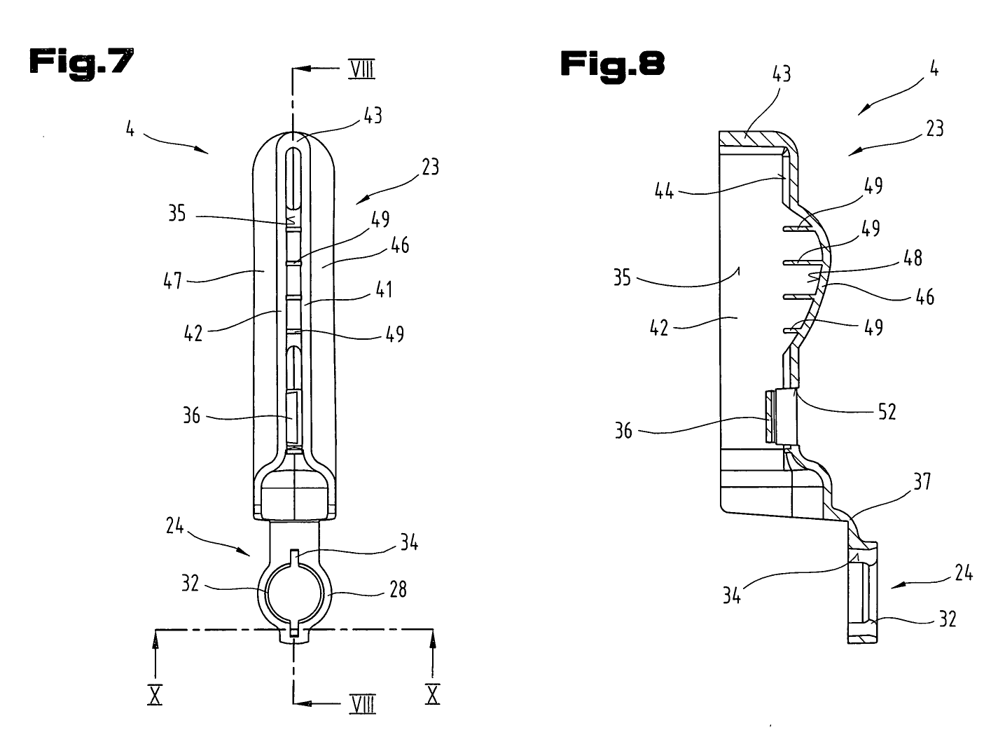

FIG. 7 is a side view of the protective device according to FIGS. 5 and 6.

FIG. 8 is a sectional view of the protective device according to FIGS. 5 to 7, with a section according to lines VIII-VIII in FIG. 7.

FIG. 9 is a sectional top view of a part area of the protective device according to FIGS. 5 to 8, with a section according to lines IX-IX in FIG. 6.

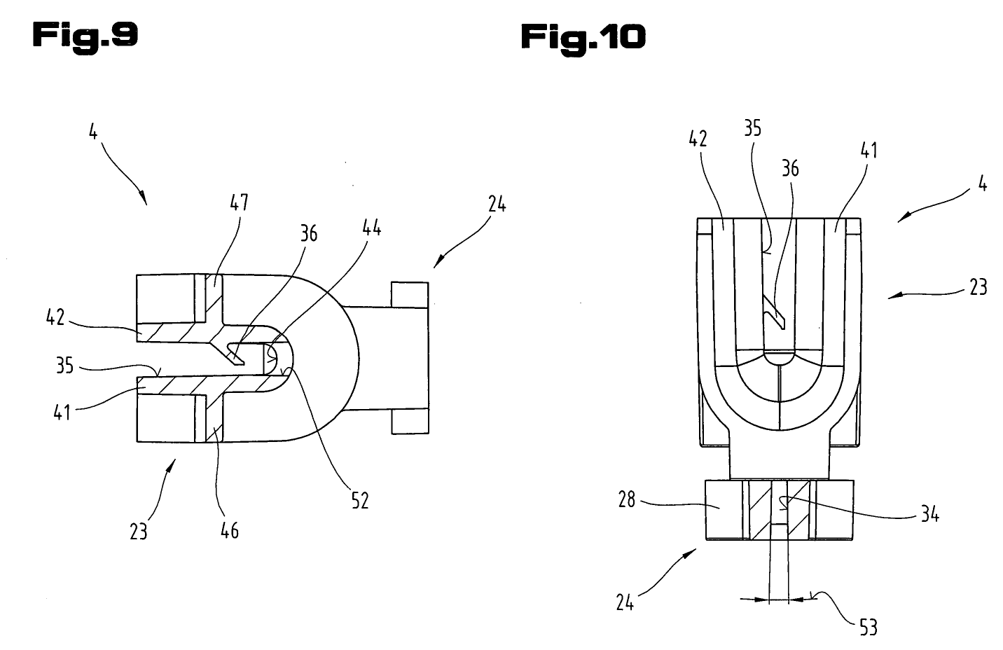

FIG. 10 is a sectional bottom view of a part area of the protective device according to FIGS. 5 to 9, with a section according to liens X-X in FIG. 7; and

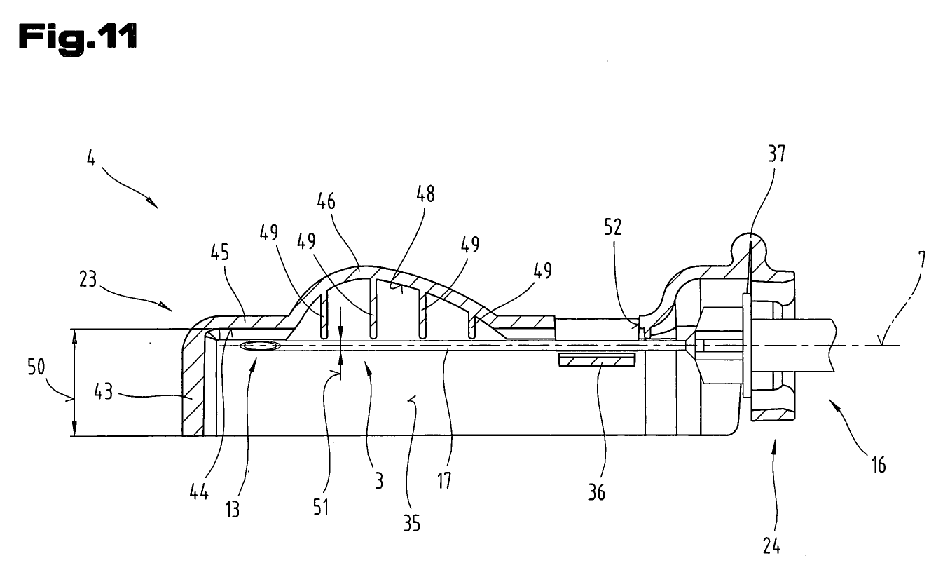

FIG. 11 is a sectional and simplified representation of the protective device in its protective position according to FIGS. 5 to 10.

It is noted herewith by way of introduction that in the different embodiments described herein, identical components are provided with identical reference number or identical component designations, whereby the disclosures contained throughout the present specification can be applied in the same sense to identical components with the same reference numbers and same component designations. Furthermore, the positional data selected in the specification such as, for example “top”, “bottom”, “lateral”, etc. relate to the directly described and shown figure, and have to be applied in the same sense to the new position where a position has changed. Moreover, individual features or combinations of features of the different exemplified embodiments shown and described herein may per se represent independent inventive solutions, or solutions as defined by the invention.

FIGS. 1 to 4 show a construction assembly 1 particularly for the medical technology, said assembly comprising at least one manipulating device 2, a puncturing element 3 that can be mounted on said manipulating device, if need be, as well as a pivot-mounted protective device 4 for at least a part area of the puncturing element 3.

The manipulating device may be formed, for example by a receiving container 6 forming or limiting a receiving chamber 5. Said receiving container has an open face side 8 as well as a face side 9 that is closed in least in a part area, said face sides being spaced from each other in the direction of a longitudinal axis 7. The face side 9 closed at least in a part area is preferably closed in the present embodiment by a face wall 10 shown here in a simplified manner. Furthermore, within the area of the at least partly closed face side, a first holding element 11 shown by a simplified representation is arranged on the manipulating device 2, and a first coupling element 12 is preferably arranged within the range of the longitudinal axis 7. In the present exemplified embodiment, the puncturing element 3 has a proximal end 13 as well as a distal end 14, said ends being spaced from each other in the direction of the longitudinal axis 7, whereby a passage 15 is connecting the two ends 13 and 14 with one another. This permit flow through the hollow puncturing element 3. It is generally pointed out here also that the designations of the two ends 13 and 14 relate to the patient, i.e. the proximal end 13 is facing the patient, and the distal end 14 is facing away from the patient.

Furthermore, between the two ends 13 and 14, another coupling element 16 is arranged fixed on a hollow needle 17 of the puncturing element 3. The first coupling element 12 on the receiving container 6 of the manipulating device 2, and the other coupling element 16 on the hollow needle 17 of the puncturing element 3, are jointly forming a coupling device 18 for coupling the puncturing element 3 to the manipulating device 2 as required. In this way, the puncturing element 3, which is referred to also as a cannula, can be mounted on the manipulating device 2, if necessary in a detachable manner.

As shown here in the present exemplified embodiment, if the puncturing element 3 has the form of a double-ended hollow needle 17, a part area of the hollow needle 17, namely the proximal end 13, protrudes over the manipulating device 2, extending to the side averted from the receiving chamber 5. Furthermore, it is shown simplified in the area of the proximal end 13 that an aperture 19 is formed in the manner known per se by slanting the hollow needle 17, which due to such slant, has approximately the shape of an ellipse or oval in relation to the longitudinal axis 7. In this way, the aperture 19 has a longer as well as a shorter opening axis 20 and 21, respectively.

The protective device 4 has at least one protective element 23 adapted for pivoting in a plane of swivel 22, whereby said protective element is adapted for pivoting from a first position (=the release position) releasing the part area of the puncturing element 3, into a second position (=the protective position) covering the part area.

For mounting or attaching the protective device 4 in a detachable manner, if need be, the latter has another holding element 24, by means of which said protective device can be held in a stationary manner and detachably, if necessary, on the first holding element 11 on the receiving container 6 of the manipulating device 2. The two holding elements 11 and 24 are forming a holding device 25. The stationary mounting of the two holding elements 11 and 24 in relation to one another means that when the two holding elements 11 and 14 are fully engaged, said two holding elements 11 and 24 are prevented from any adjustment or rotation around the longitudinal axis 7, as well as from any axial movement in the direction of the longitudinal axis 7, i.e. such an adjustment or rotation and axial movement is not possible. However, if the protective device 4, particularly the holding element 24 is mounted on the first holding element 11, if need be, the pivot-mounted protective device 4 can be removed or separated again from the manipulating device 2, if necessary. The holding elements 11 and 24 may be designed in relation to each other in such a way that their reciprocal holding engagement is realized in the form of a fixed or press fit; a cone connection or a locking connection.

For achieving the relative fixation of the position or positioning of the two holding elements 11 and 24 in relation to one another, a positioning device 26 is arranged between said two holding elements 11 and 24 of the holding device 25, whereby said positioning device, when the holding elements 11, 24 engage one another, fixes the position of the plane of swivel 22 of the protective element 23 of the pivot-mounted protective device 4 in relation to the manipulating device 2.

If the first holding element 11 on the receiving container 6 of the manipulating device 2 is formed by an approximately tubular first component 27, the center axis of said component 27 may be extending aligned coincident with the longitudinal axis 7. In most cases, the first tubular component 27 is arranged fixed, extending concentrically with the longitudinal axis 7 on the receiving container 6 in the area of the closed face side 9 on the face wall 10, said face side being closed at least in areas. Such a center or central arrangement with respect to the receiving container 6 is used in connection with a double-ended puncturing element 3, whereby as described above, the proximal end 13 is then protruding beyond the face wall 10 in the direction away from the receiving chamber 5, and the distal end 14, when positioned on the manipulating device 2, then projects into the direction of the open face side 8. Such an arrangement of the puncturing element 3 with respect to the manipulating device 2 is generally known in this form.

The first holding element 11 or the tubular component 27 forming said element projects beyond the face wall 10 to the side facing away from the receiving chamber 5. The other holding element 24 of the protective device 4- may be formed by another approximately tubular component 28 as well, whereby the latter is extending beyond the first tubular component 27 in the area of the outer surface of the latter, particularly in the area of its cylinder jacket, at least in part areas. Thus the outer dimension or the outside diameter of the first holding element 11 approximately corresponds with the inside dimension or inside diameter of the other holding element 24, so that due to the selected dimensions, the other holding element 24 can be axially pushed over or mounted on the first holding element 11. In order to prevent the two holding elements 11, 24 from getting unintentionally detached from each other when in the position in which they are engaged, the holding device 25 may comprise the additional locking elements 29, 30 adapted for engaging each other. Such locking elements can be seen in the best way by jointly viewing the FIGS. 3 and 4. Said two locking elements 29, 30 may be arranged in least in part areas over the periphery of the two holding elements 11, 24, or their components 27, 28 on the sides facing one another.

The two holding elements 11 and 24, or their components 27 and 28, respectively, may naturally have any three-dimensional shape such as, e.g. an oval, multi-corned, etc. form. However, the positioning device 26 for holding the protective element 4 in an oriented manner on the manipulating device 2 may be formed by such holding elements at the same time by designing the latter accordingly.

In the exemplified embodiment shown in said figures, the first locking element 29 (see FIG. 3) is formed on the first holding element 11, namely the tubular component 27 shown there by a bead 31 projecting beyond said component 27 on the side facing away from the longitudinal axis 7. Said bead may be arranged extending at least in part areas, but also continuously over the periphery of the holding element 11.

The other locking element 30 is formed on the holding element 24, for example the tubular component 28 shown here, by another bead 32 projecting beyond the side facing the longitudinal axis 7. This is shown most clearly in FIG. 4. Said bead 32 forming the locking element 30 may again be arranged at least in sections across the inner periphery of the holding element 24, in the present case the tubular component 28, whereby in the locking position, the other locking element 30 is extending on the side facing the face wall 10 over the locking element 29 arranged on the first holding element 11.

What is achieved with this embodiment, which is described only by way of example for a great number of other possible embodiments of the cooperating locking elements 29 and 30, is that with the holding elements 11 and 24 in engaged positions, any unintentional movement of said two holding elements 11 and 24 in relation to each other in the direction of the longitudinal axis 7 is prevented. However, because of the design of the cooperating locking elements described above, it is possible for the protective device 4 to swivel or rotate relative to the manipulating device 2 around the longitudinal axis 7 if the manipulating device 2 is held in a stationary manner. However, the locking element 29 naturally may be formed also, for example by one or more groove-like recesses on the component 27, and the other locking element 30 by one or more projections on the other component 28, such projections cooperating with or engaging said recesses.

As already described above, the positioning device 26 is arranged between the two holding elements 11 and 24 of the holding device 25. The position of the protective element 23 of the protective device 4, and connected therewith the plane of swivel 22 are fixed by such an arrangement vis-á-vis the manipulating device 2. The positioning device 26 may be realized in all sorts of different ways. In the exemplified embodiment shown here, the positioning device is formed within the area of the manipulating device 2 by at least one first positioning element 33, which is aligned in the direction of the longitudinal direction 7 as well as perpendicularly to the latter, and within the area of the other holding element 24 of the protective device 4 by at least one other positioning element 34, which is approximately formed as a counterpart to the first positioning element.

It is shown most clearly in FIG. 2 that the first positioning element 33 is formed in the present case by a rib or bridge, which is arranged in the area of the outer surface of the first holding element 11, namely the tubular component 27, projecting radially beyond the side facing away from the longitudinal axis 7. In order to permit simple mounting of the two holding elements 11 and 24, the first positioning element 33 is arranged extending in the direction of the longitudinal axis 7. The other positioning element 34 may be formed within the area of the other holding element 24, for example by a recess or slot formed as a counterpart for the positioning element 33.

It is, of course, possible also to form the positioning element 26 on the other holding element 24 of the protective device 4 by positioning elements 24 diametrically opposing each other, particularly like the recesses or slots described above. In cooperation with the first positioning element 33 on the manipulating device 2, the protective device 4 can then be mounted on the receiving container 6 of the manipulating device 2 turned by 180°.

FIG. 2 furthermore shows in a simplified way that the protective element 23 has a duct 35 for receiving at least a part area on the puncturing element 3, namely the hollow needle 15 within the area of its proximal end 13. In order to prevent the protective element from pivoting back from the protective position, at least one retaining element 36 is arranged in the area of the duct 35 for holding it locked in its protective position on the part area of the puncturing element 3, in a manner known already from the prior art.

For achieving the pivotal movement of the protective element 23 in relation to the other holding element 24, it is possible to arrange an elastically deforming bridge between said components. For exactly fixing the pivotal movement and the position of the protective element 23, in its protective position covering the part area of the puncturing element 3, it is advantageous if the protective element 23 is capable of swiveling around an axis of swivel 37 aligned perpendicularly to the plane of swivel 22, whereby said axis of swivel 37 may be preferably formed by a film hinge.

The position of the plane of swivel 22 relative to the receiving container 6 of the manipulating device 2 is fixed by the cooperation of the holding elements 11 and 24 engaging one another, as well as in conjunction with the cooperating locking elements 29 and 30, if need be. In most cases, the plane of swivel 22 is aligned extending parallel to or on the longitudinal axis 7.

As already briefly described above, the coupling device 18 is formed between the puncturing element 3 and the manipulating device 2, particularly the receiving container 6, said coupling device comprising the first coupling element 12 arranged on the receiving container 6, and the other coupling element 16 arranged on the hollow needle 17 of the puncturing element 3. It is important in this connection that the two coupling elements 12 and 16 of the coupling device 18 are designed or aligned in relation to one another in such a way that when the two coupling elements 12 and 16 are in the position in which they are completely coupled with each other, the shorter opening axis 21 of the aperture 19 is aligned extending approximately parallel to the plane of swivel 22 or in the plane of swivel 22. Now, viewing the position of the plane of swivel 22 as fixed by the positioning device 26 with respect to the receiving container 6 of the manipulating device 2, with an aperture 19 at the proximal end 13 facing and being visible to the user of the medical assembly 2, the protective element 23 is arranged in the release position with respect to the manipulating device 2 either on the left or right of the part area of the hollow needle 17 to be covered. Due to such mutual alignment, with the two coupling elements 12 and 16 being in the position in which they are completely coupled with one another, a visual contact with the approximately elliptical or oval aperture 19 is always possible, as it is necessary when the manipulating device, the protective device and the medical assembly 1 formed by said devices is employed for drawing body fluids and/or dispensing liquid substances into the body of a patient for medical purposes. The manipulating device 2, the protective device 4 or the medical assembly 1 formed by said devices are mostly employed in the medical field of blood letting.

In the present embodiment, it is also shown that in the area of the manipulating device 2, the first coupling element 12 and the first holding element 11 are arranged concentrically with each other on the face side 9 or face wall 10, said face side being closed at least in part areas. Due to the tubular component 27 described above, which is forming the holding element 11, the coupling device 18 may be formed by an arrangement of threads engaging one another, whereby the first coupling element 12 on the tubular component 27 may be formed by a female thread, and a male thread engaging said female thread may be formed on the other coupling element 16 of the puncturing element 3. The thread arrangement is preferably formed by a double thread, so that an application possibility is achievable and connected therewith an end position of the aperture 19 on the puncturing element 3 that is offset or turned in each case by 180° relative to the plane of swivel 22. Owing to the arrangement and realization of the coupling elements 12, 16 in relation to one another as described above, it is always possible to see the shorter opening axis 21 of the aperture 19 either from the front or rear viewing side of the plane of swivel 22.

Furthermore, it is ensured in this way that the protective element 23 cannot be pivoted at an angle relative to the opening axis 21, so that for example during a drawing of blood, the protective element 23, when used as intended, is not facing the arm of the patient and thus hindering the letting of blood. With the protective element 23 in a position that has been turned by about 180°, said element prevents the aperture 19 from being seen, which is required for carrying out a puncture of a body part such as, for example a vein or artery, so that the puncture can be executed as intended.

However, instead of using the thread arrangement for forming the coupling elements 12, 16, it is possible also to realize the coupling device 18, for example in the form of a bayonet arrangement or the like. Also, the coupling device 18 may be formed by a cone connection, in connection with which it is again necessary to ensure that in the coupled position, the shorter opening axis 21 of the aperture 19 is aligned extending approximately parallel to the plane of swivel 22, or in the latter. This can be achieved by means of an additional positioning device arranged between the components to be engaged.

It is advantageous in connection with said medical assembly 1 that the protective device can be pre-mounted already on the manipulating device independently of the puncturing element 3 and has to be attached to the manipulating device 3 when the puncturing element 3 is used as intended. In the present case, it is screwed into the manipulating device. As it is being used employed as intended, the protective element 23 is arranged laterally of the manipulating device 2 in the plane of swivel 22 without assuming an obstructive position either between the arm of the patient and the manipulating device 2, or blocking the visual contact with the aperture 19 of the puncturing element 3 facing the user or, e.g. a physician.

In order to achieve a superior fixed seating of the two cooperating holding elements 11 and 24, or also to prevent said two elements from getting unintentionally detached from one another, it may be advantageous if, in a plane aligned perpendicularly to the longitudinal axis 7, a stop component 38 of the other coupling element 16, such a step means facing the face wall 10 of the receiving container 6, has a larger inside dimension 39 vis-á-vis the inside dimension 40 of the other holding element 24. In this way, the stop component 38 will then extend at least beyond a part area of the tubular component 28, and in this way prevents the protective device 4 from being inadvertently removed from the manipulating device 2.

The manipulating device 2, the protective device 4 and the coupling element 16 on the puncturing element 3 are manufactured from a plastic in most cases, whereby the manipulating device 2 is preferably transparent, so that the interior space can be viewed. It is of importance in connection with said assembly or the components forming the latter that the puncturing element 3, e.g. the cannula with its aperture 19 is fixed oriented with respect to the coupling element 16 and the protective device 4, or that it is fixed in its position on the manipulating device 2 with respect to the coupling element 12.

FIGS. 5 to 11 show another embodiment of the protective device 4 for forming the medical assembly 1. Said embodiment may be independent per se, if need be. The same reference numbers and component designations are again used for identical components as in the preceding FIGS. 1 to 4. Furthermore, in order to avoid unnecessary repetitions, reference is made to the detailed description of the preceding FIGS. 1 to 4.

The present protective device 4, too, serves for covering as required the puncturing element 3, which is shown here only in part, in cooperation with the manipulating device 2. Said protective device 4 again comprises the protective element 23, the other holding element 24 for holding or coupling with the first holding element 11 on the manipulating device 2 by means of the holding device 25 forming said holding elements.

The holding element 24 also comprises the locking element 30 described already in detail above, as well as the positioning element 34. The protective element 23 is again adapted for pivoting via the swivel axis 37 in the plane of swivel 22, starting from the position (=release position) releasing the part area of the puncturing element 3, into the second position (=the protective position) covering the part area. In the present exemplified embodiment, the duct 35 is limited across its longitudinal expanse by the bridges 41 and 42, which are extending parallel to each other and perpendicularly to the longitudinal expanse, and which are spaced from each other. Furthermore, the duct 35 is limited on side facing away from the other holding element 24 by a face wall 43, which connects the two bridges with each other on the side facing away from the other holding element. 24. By jointly viewing the FIGS. 5 and 7, it can be seen that within the near range of the other holding element 24, the two bridges 41 and 42 are farther spaced from each other than in their further longitudinal expanse. Such widening of the duct 35 serves the purpose of receiving in a protected manner a part of the other coupling element 16 (see the representation in FIG. 11), and to cover said part at the same time.

By jointly viewing the FIGS. 8 and 11, it is most clearly visible that at least one retaining element 36 is again arranged in the duct 35, which, in the present exemplified embodiment, is arranged in about the first half of the longitudinal expanse of the duct 35, starting from the other holding element 24. The retaining element 36 is preferably arranged in about a third part of the total longitudinal expanse of the duct 35, starting from the other holding element 24. However, it is possible also that the retaining element 36, in its position (=covering position) where it is covering the protective element 23, is arranged in the section between the other holding element 24 of the protective device 4, and half of the distance between the proximal end 13 and the other holding element 24.

For locking the protective device 4, particularly the protective element 23 in a superior manner on the part of the puncturing element 3 to be covered, the retaining element 36 has a longitudinal expanse in the direction of the duct 35 between 3 mm and 10 mm, preferably between 5 mm and 7 mm. Such larger longitudinal expanse of the retaining element 36 serves the purpose of achieving superior guidance and fixation of the position where the protective element 23 is in its locked position on the puncturing element 3.

Within the area of the bottom 44 of the duct 35, the protective element 23 has a wall component 45 connecting one of the two bridges 41, 42 at least by sections. In this way, the duct 35 is now covering or surrounding the puncturing element 3 to be covered on at least four sides when it is in the covering position (=protected position).

Furthermore, it is advantageous if a gripping bar 46, 47 is protruding over at least one of the two bridges 41, 42 in the vertical direction relative to the bridge on the side facing away from the duct 35. The gripping bars 46, 47 are preferably arranged symmetrically in relation to the bridges 41, 42 and, on the side facing away from the duct 35, project over the two bridges 41, 42 in the vertical direction relative to said bridges. For superior handling, the two gripping bars 46, 47 each have a curved contour, viewed in each case across their longitudinal expanse, as it is most clearly visible in FIGS. 5 and 6.

Furthermore, the representations of FIGS. 8 and 11 show that the duct 35 has an additional concave recess 48 between the retaining element 36 and the face wall 43, so that the duct 35 has a recessed duct bottom 44. Said additional concave recess 48 serves the purpose of enhancing the handling of the protective element 23 within the area of the outer surface, on the one hand, and of providing a receiving space for fluids that possibly might exit from the puncturing element 3 on the other.

In the present exemplified embodiment, within the area of the concave recess 48, one of the gripping bars 46, 47 is limiting the duct 35 within the area of its recessed duct bottom 44. For the purpose of achieving superior support and guidance of the section of the puncturing element 3 that has to be received in the duct 35, at least one repelling bridge 49 is arranged in the area of the concave recess 48, whereby the one or more repelling bridges 49 are extending between the two bridges 41 and 42 and are connecting the latter with each other. In this connection, the one or more repelling bridges 49 are aligned in the vertical direction relative to the bridges 41 and 42, as well as in the vertical direction in relation to the longitudinal expanse of the duct 35, if necessary. Furthermore, the one or more repelling bridges 49 are ending in the area of the bottom 44 of the duct 35, starting from the concave recess 48.

Now, it is most clearly shown in FIG. 11 that in the covering position (=protective position), the bottom 44 of the duct is extending directly adjacent to the longitudinal axis 7 extending through the other holding element 24, so that the part of the puncturing element 3 to be covered is arranged directly adjacent to the bottom 44 of the duct. In order to prevent the proximal end 13 of the puncturing element 3 from exiting from the duct 35 in the event the protective element 23 is improperly manipulated, the depth 50 of the duct 35, in the vertical direction relative to the longitudinal expanse, amounts to a multiple such as, e.g. from 5 times up to 20 times the outside dimension 51 of the part of the puncturing element 3 to be covered. This ensures that even if an attempt were made to move the protective element 23 against the closing movement from the locked position and away from the puncturing element 3, the proximal end 13 and thus its tip posing a high risk of causing injury, are safely covered in any case. The depth 50 of the duct is highly dependent upon the outside dimension 51 and may also amount to 10 to 15 times said dimension.

For a superior design of the retaining element 36, it is advantageous if the wall component 45 of the duct 35 limiting the bottom 44 of the latter has an aperture 52 within the area of the retaining element 36. As shown most clearly in FIGS. 9 and 10, the retaining element 36 is arranged within the duct 35 on one of the bridges, in the present exemplified embodiment on the bridge 42, and is formed in such a manner that starting from said bridge, it is protruding into the duct 35 in the direction of the bottom 44 of the latter. The retaining element 36, due to its position extending at an angle, and the wall thickness selected for it, has certain properties of elastic deformation in order to permit the puncturing element 3 to pass through to the bottom 44 of the duct between the end of the holding element 23 and the bridge 41 disposed opposite said holding element. So as to avoid unlocking or release of the protective element 23 from its arrested position, the retaining element 36 possesses adequate rigidity in the opposite direction of actuation, whereby the distance between the end of the retaining element 36 and the opposite bridge 41 is always reduced when another opening movement occurs.

As described already above, the protective element 23 is flexibly connected with the other holding element 24 by means of the film hinge acting as the swivel axis 37, whereby prior to the first adjustment or setting to the covering position (=protective position), the film hinge is forming a fixation of the position for the protective element that can be overcome due to the inherent properties of the film or sheet material.

Finally, the representation of FIG. 10 also shows that the position element 34, which has the form of a gap or slot in the present embodiment, has limiting walls that are constantly narrowing by an angle 53, starting from the part of the holding element 24 that has to be facing the manipulating device 2, so when the holding element 24 is placed on the first holding element 11 of the manipulating device 2, superior fixation of the position can be achieved with the bridge- or rib-like positioning element 33 arranged in said site.

However, independently of the foregoing, the assembly described above can be employed not only in the field of medical technology for drawing blood and collecting other body fluids (urine and other secreted substances), but used also for collecting and receiving specimens in all kinds of different application areas such as, e.g. for hazardous substances, chemical products, etc.

In the interest of good order it is finally pointed out that for the sake of better understanding of the structure of the medical assembly, the latter and its components are partly represented untrue to scale and/or enlarged and/or reduced.

Most of all, the individual embodiments shown in FIGS. 1, 2; 3; 4; and 5 to 11 form the object of independent inventive solutions. The problem forming the basis of such independent inventive solutions are specified in the description.

### LIST OF REFERENCE NUMBERS

1 Assembly

2 Manipulating device

3 Puncturing element

4 Protective device

5 Receiving chamber

6 Receiving container

7 Longitudinal axis

8 Face side

9 Face side

10 Face wall

11 Holding element

12 Coupling element

13 Proximal end

14 Distal end

15 Passage

16 Coupling element

17 Hollow needle

18 Coupling device

19 Aperture

20 Opening axis

21 Opening axis

22 Plane of swivel

23 Protective element

24 Holding element

25 Holding device

26 Positioning device

27 Component

28 Component

29 Locking element

30 Locking element

31 Bead

32 Bead

33 Positioning element

34 Positioning element

35 Duct

36 Retaining element

37 Axis of swivel

38 Stop component

39 Dimension

40 Dimension

41 Bridge

42 Bridge

43 Face wall

44 Bottom of duct

45 Wall component

46 Gripping bar

47 Gripping bar

48 Recess

49 Repelling bridge

50 Depth of duct

51 Outside dimension

52 Aperture

53 Angle

1-68. (canceled)

69. An assembly (1) particularly for the medical technology, comprising a manipulating device (2) with a first holding element (11) and a first coupling element (12) arranged thereon, a puncturing element (3) with a proximal end and a distal end (13, 14), said ends being spaced from one another in the direction of a longitudinal axis (7), whereby a passage (15) is connecting the two ends (13, 14) with each other; with another coupling element (16) arranged between the ends (13, 14), whereby the two coupling elements (12, 16) form a coupling device (18), and the puncturing element (3) is mounted, if necessary detachably, on the manipulating device (2), whereby the proximal end (13) of the puncturing element (3) protrudes beyond the manipulating device (2), and an aperture (19) with a longer and a shorter opening axis (20, 21) is arranged at least on the proximal end (13), and with a pivot-mounted protective device (4) for at least a part area or section of the puncturing element (3), said protective device (4) being mounted stationarily, if necessary detachably with another holding element (24) on the first holding element (11) of the manipulating device (2), whereby the two holding elements (11, 24) form a holding device (25), and one protective element (23) of the protective device (4) is adapted for pivoting in a plane of swivel (22) aligned extending in the longitudinal axis (7), from a first position (release position) releasing the part area or section of the puncturing element (3), into a second position (protective position) covering said part area or section, wherein a positioning device (26) is arranged between the two holding elements (11, 24) of the holding device (25), said positioning device, with the holding elements (11, 24) being in their engaged positions, fixing the position of the plane of swivel (22) of the protective element (23) of the protective device (4) with respect to the manipulating device (2); and in that the two coupling elements (12, 16) of the coupling device (18) are designed relative to one another in such a way that with the two coupling element (12, 16) being fully engaged, the shorter opening axis (21) of the aperture (19) is aligned extending about parallel to the plane of swivel (22) or in the plane of swivel (22).

70. The assembly (1) according to claim 69, wherein the manipulating device (2) is formed by at least one receiving container (6) with a face side (8, 9) closed at least in sections by a face wall (10), said receiving container forming a receiving chamber (5).

71. The assembly (1) according to claim 69, wherein the first coupling element (12) and the first holding element (11) are arranged concentrically with one another on the at least partly closed face side (9).

72. The assembly (1) according to claim 69, wherein the first holding element (11) is formed by an approximately tubular first component (27).

73. The assembly (1) according to claim 72, wherein the tubular component (27) protrudes over the face wall (10) on the side averted from the receiving chamber (5).

74. The assembly (1) according to claim 69, wherein the other holding element (24) is formed by another approximately tubular component (28), said component protruding over the outer surface of the first tubular component (27) at least by sections.

75. The assembly (1) according to claim 69, wherein the holding device (25), furthermore, comprises locking elements (29, 30) adapted for engaging each other, said locking elements being arranged on the two holding elements (11, 24) at least by sections.

76. The assembly (1) according to claim 75, wherein the first locking element (29) on the first holding element (11) is formed by a bead (31) protruding over the tubular component (27) to the side averted from the longitudinal axis (7), said bead being arranged extending over the periphery at least by sections.

77. The assembly (1) according to claim 75, wherein the other locking element (30) on the other holding element (24) is formed by another bead (32) protruding over the tubular component (28) to the side averted from the longitudinal axis (7), said bead being arranged extending over the periphery at least by sections, and with the other locking element (30) being in the locking position, protruding over the first locking element (29) on the side facing the face wall (10).

78. The assembly (1) according to claim 69, wherein the protective element (23) is adapted for pivoting around an axis of swivel (37) aligned perpendicularly to the plane of swivel (22).

79. The assembly (1) according to claim 78, wherein the axis of swivel (37) is formed by a film hinge.

80. The assembly (1) according to claim 79, wherein prior to the first setting to the covering position (protective position), the film hinge forms a surmountable fixation of the position for the protective element (23).

81. The assembly (1) according to claim 69, wherein the coupling device (18) is formed by an arrangement of threads engaging one another.

82. The assembly (1) according to claim 81, wherein the arrangement of threads is formed by a double thread.

83. The assembly (1) according to claim 69, wherein in a plane aligned perpendicularly to the longitudinal axis (7), a stop component (38) of the other coupling element (16), said stop facing the face wall (10), has a larger dimension (39) vis-á-vis the inside dimension (40) of the other holding element (24).

84. The assembly (1) according to claim 69, wherein with the positioning device (26) is formed within the area of the manipulating device (2) by at least one first positioning element (33) aligned extending in the direction of the longitudinal axis (7) as well as perpendicularly to said axis (7), and within the area of the other holding element (24) by at least one other positioning element (34) shaped in the form of an approximately identical counterpart to said other holding element.

85. The assembly (1) according to claim 84, wherein the first positioning element (33) is formed by a rib or a bridge.

86. The assembly (1) according to claim 84, wherein the first positioning element (33) is formed on the tubular component (27) of the first holding element (11) and protrudes over the latter to the side averted from the longitudinal axis (7).

87. The assembly (1) according to claim 84, wherein the other positioning element (34) is formed by a recess or a slot.

88. The assembly (1) according to claim 69, wherein the protective element (23) of the protective device (4), in the position in which it is covering the puncturing element (3) at least by sections, is retained locked on the puncturing element (3).

89. The assembly (1) according to claim 69, wherein the protective element (23) has a duct (35) for receiving at least a part area or section of the puncturing element (3).

90. The assembly (1) according to claim 89, wherein in the covering position (protective position), the duct (35) covers at least four sides of the puncturing element (3) to be covered.

91. The assembly (1) according to claim 88, wherein at least one retaining element (36) is arranged in the duct (35) for holding the part area or section of the puncturing element (3) locked in the protective position.

92. The assembly (1) according to claim 88, wherein in the covering position (protective position) of the protective element (23), the retaining element (36) is arranged in the section extending between the other holding element (24) of the holding device (4), and half of the distance between the proximal end (13) and the other holding element (24).

93. The assembly (1) according to claim 92, wherein the retaining element (36) is arranged in about a third part of the total longitudinal expanse of the duct (35), starting from the other holding element (24).

94. The assembly (1) according to 89, wherein the duct (35) by a face wall (43) on the side averted from the other holding element (24).

95. The assembly (1) according to claim 89, wherein the duct (35) is limited over its longitudinal expanse by bridges (41, 42), the latter being spaced from one another and extending parallel to one another and perpendicularly to the longitudinal expanse.

96. The assembly (1) according to claim 95, wherein the bridges (41, 42) are farther spaced from one another within the near range of the other holding element (24) than in their further longitudinal expanse.

97. The assembly (1) according to claim 95, wherein the two bridges (41, 42) are connected with each other via the face wall (43) on the side averted from the other holding element (24).

98. The assembly (1) according to claim 89, wherein the duct (35) has an additional concave recess (48) between the retaining element (36) and the face wall (43).

99. The assembly (1) according to claim 95, wherein on the side averted from the duct (35), a gripping bar (46, 47) protrudes over at least one of the bridges (41, 42) in the vertical direction.

100. The assembly (1) according to claim 99, wherein the gripping bars (46, 47) protrude over the two bridges (41, 42) to the side averted from the duct (35).

101. The assembly (1) according to claim 99, wherein that the gripping bar (46, 47) has a curved longitudinal contour over its longitudinal expanse.

102. The assembly (1) according to claim 99, wherein the gripping bar (46, 47) limits the duct (35) within the area of the concave recess (48).

103. The assembly (1) according to claim 98, wherein at least one repelling bridge (49) is arranged in the duct (35) within the area of the concave recess (48), said repelling bridge supporting and guiding the part of the puncturing element (3) to be received in the duct (35).

104. The assembly (1) according to claim 103, wherein the one or more repelling bridges (49) extend between the two bridges (41, 42), connecting the latter with one another.

105. The assembly (1) according to claim 103, wherein the one or more repelling bridges (49) are aligned perpendicularly to the bridges (41, 42).

106. The assembly (1) according to claim 103, wherein the one or more repelling bridges (49) are aligned perpendicularly to the longitudinal expanse of the duct (35).

107. The assembly (1) according to claim 103, wherein the one or more repelling bridges (49) end within the area of the bottom (44) of the duct (35), starting from the concave recess (48).

108. The assembly (1) according to claim 88, wherein in the covering position (protective position) of the protective element (23), the covered part of the puncturing element (3) is disposed within the area of the bottom (44) of the duct (35).

109. The assembly (1) according to claim 89, wherein the depth (50) of the duct (35) in the direction perpendicular to the longitudinal expanse of said duct amounts to a multiple such as to 5 times to 20 times of the outside dimension (51) of the part of the puncturing element (3) to be covered.

110. The assembly (1) according to claim 89, wherein the bottom (44) of the duct (35) has an opening (52) within the area of the retaining element (36).

111. The assembly (1) according to claim 91, wherein the retaining element (36) is arranged on one of the bridges (41, 42) within the duct (35), and protrudes starting from said duct (35) into the latter in the direction of the bottom (44) of the duct (35).
